(19) United States
(12) Patent Application Publication
(10) Pub. No.: US 2006/0013892 A1
(43) Pub. Date: Jan. 19, 2006

(54) Administration of amino acid chelates for reduction in alcohol dependency

(75) Inventor: H. DeWayne Ashmead, Fruit Heights, UT (US)

Correspondence Address: THORPE NORTH & WESTERN, LLP., 8180 SOUTH 700 EAST, SUITE 200, SANDY, UT, 84070, US

(73) Assignee: Albion International Inc.

(21) Appl. No.: 11/173,956

(22) Filed: Jul. 1, 2005

Related U.S. Application Data
(63) Continuation-in-part of application No. 10/306,711, filed on Nov. 26, 2002, now abandoned.
(60) Provisional application No. 60/334,051, filed on Nov. 28, 2001.

(51) Int. Cl. A61K 31/198; A61K 33/00

(52) U.S. Cl. 424/600; 514/566

(57) ABSTRACT

A method for reducing alcohol desire or dependency in a human can comprise the steps of administering an amino acid chelate or a combination of amino acid chelates to a human having alcohol dependency symptoms or an unwanted desire for alcohol. Naturally occurring amino acids may be used as ligands to form the chelates. Metals that can be used include nutritionally relevant metals, including copper, zinc, and manganese.

The present application is a Continuation-In-Part of U.S. patent application Ser. No. 10/306,71 1, filed on Nov. 26, 2002, which claims the benefit of U.S. Provisional Application No. 60/334,051 filed on Nov. 28, 2001, both of which are incorporated herein by reference which is incorporated herein by reference in its entirety.

### FIELD OF THE INVENTION

The present invention is drawn to methods for reducing alcohol dependency. More specifically, the present invention is drawn to the use of certain amino acid chelates or combinations of amino acid chelates to reduce a dependency or desire for consumption of alcohol in humans.

### BACKGROUND OF THE INVENTION

Amino acid chelates are generally produced by the reaction between a-amino acids and metal ions having a valence of two or more to form a ring structure. In such a reaction, the positive electrical charge of the metal ion can be neutralized by the electrons available through the carboxylate or free amino groups of the a-amino acid.

Traditionally, the term “chelate” has been loosely defined as a combination of a metallic ion bonded to one or more ligands to form a heterocyclic ring structure. Under this definition, chelate formation through neutralization of the positive charge(s) of the metal ion may be through the formation of ionic, covalent or coordinate covalent bonding. An alternative and more modern definition of the term “chelate” requires that the metal ion be bonded to the ligand solely by coordinate covalent bonds forming a heterocyclic ring. In either case, both are definitions that describe a metal ion and a ligand forming a heterocyclic ring.

Chelation can be confirmed and differentiated from mixtures of components by infrared spectra through comparison of the stretching of bonds or shifting of absorption caused by bond formation. As applied in the field of mineral nutrition, there are certain “chelated” products that are commercially utilized. The first is referred to as a “metal proteinate.” The American Association of Feed Control officials (AAFCO) has defined a “metal proteinate” as the product resulting from the chelation of a soluble salt with amino acids and/or partially hydrolyzed protein. Such products are referred to as the specific metal proteinate, e.g., copper proteinate, zinc proteinate, etc. Sometimes, metal proteinates are even referred to as amino acid chelates, though this characterization is not completely accurate.

The second product, referred to as an “amino acid chelate,” when properly formed, is a stable product having one or more five-membered rings formed by a reaction between the amino acid and the metal. The American Association of Feed Control Officials (AAFCO) have also issued a definition for amino acid chelates. It is officially defined as the product resulting from the reaction of a metal ion from a soluble metal salt with amino acids having a mole ratio of one mole of metal to one to three (preferably two) moles of amino acids to form coordinate covalent bonds. The average weight of the hydrolyzed amino acids must be approximately 150 and the resulting molecular weight of the chelate must not exceed 800. The products are identified by the specific metal forming the chelate, e.g., iron amino acid chelate, copper amino acid chelate, etc.

In further detail with respect to amino acid chelates, the carboxyl oxygen and the a-amino group of the amino acid each bond with the metal ion. Such a five-membered ring is defined by the metal atom, the carboxyl oxygen, the carbonyl carbon, the a-carbon and the a-amino nitrogen. The actual structure will depend upon the ligand to metal mole ratio and whether the carboxyl oxygen forms a coordinate covalent bond or an ionic bond with the metal ion. Generally, the ligand to metal molar ratio is at least 1:1 and is preferably 2:1 or 3:1. However, in certain instances, the ratio may be 4:1. Most typically, an amino acid chelate with a divalent metal can be represented at a ligand to metal molar ratio of 2:1 according to Formula 1 as follows:

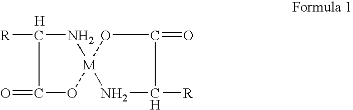

In the above formula, the dashed lines represent coordinate covalent bonds, covalent bonds, or ionic bonds. Further, when R is H, the amino acid is glycine, which is the simplest of the a-amino acids. However, R could be representative of any other side chain that, when taken in combination with the rest of the ligand structure(s), results in any of the other twenty or so naturally occurring amino acids derived from proteins. All of the amino acids have the same configuration for the positioning of the carboxyl oxygen and the a-amino nitrogen with respect to the metal ion. In other words, the chelate ring is defined by the same atoms in each instance, even though the R side chain group may vary.

With respect to both amino acid chelates and metal proteinates, the reason a metal atom can accept bonds over and above the oxidation state of the metal is due to the nature of chelation. For example, at the a-amino group of an amino acid, the nitrogen contributes to both of the electrons used in the bonding. These electrons fill available spaces in the d-orbitals forming a coordinate covalent bond. Thus, a metal ion with a normal valency of +2 can be bonded by four bonds when fully chelated. In this state, the chelate is completely satisfied by the bonding electrons and the charge on the metal atom (as well as on the overall molecule) is zero. As stated previously, it is possible that the metal ion can be bonded to the carboxyl oxygen by either coordinate covalent bonds or ionic bonds. However, the metal ion is preferably bonded to the a-amino group by coordinate covalent bonds only.

The structure, chemistry, bioavailability, and various applications of amino acid chelates are well documented in the literature, e.g. Ashmead et al., Chelated Mineral Nutrition, (1982), Chas. C. Thomas Publishers, Springfield, Ill.; Ashmead et al., Intestinal Absorption of Metal Ions, (1985), Chas. C. Thomas Publishers, Springfield, Ill.; Ashmead et al., Foliar Feeding of Plants with Amino Acid Chelates, (1986), Noyes Publications, Park Ridge, N.J.; U.S. Pat. Nos. 4,020,158; 4,167,564; 4,216,143; 4,216,144; 4,599,152; 4,725,427; 4,774,089; 4,830,716; 4,863,898; 5,292,538; 5,292,729; 5,516,925; 5,596,016; 5,882,685; 6,159,530; 6,166,071; 6,207,204; 6,294,207; 6,614,553; each of which are incorporated herein by reference.

One advantage of amino acid chelates in the field of mineral nutrition is attributed to the fact that these chelates are readily absorbed from the gut and into mucosal cells by means of active transport. In other words, the minerals can be absorbed along with the amino acids as a single unit utilizing the amino acids as carrier molecules. Therefore, the problems associated with the competition of ions for active sites and the suppression of specific nutritive mineral elements by others can be avoided.

### SUMMARY OF THE INVENTION

It has been recognized that the use of certain amino acid chelates or combinations of amino acid chelates can reduce alcohol desire and/or dependency in humans. In one embodiment, a method for reducing alcohol desire or dependency in a human can comprise the steps of administering an amino acid chelate or a combination of amino acid chelates to a human having alcohol dependency symptoms or an unwanted desire for alcohol, wherein the amino acid chelate(s) comprises a naturally occurring amino acid ligand and a metal selected from the group consisting of copper, zinc, and manganese, and wherein the amino acid to metal molar ratio is from 1:1 to 4:1.

Additionally, a composition for reducing alcohol dependency in humans can comprise a blend of a first amino acid chelate and a second amino acid chelate, wherein the first amino acid chelate and the second amino acid chelate each comprise a different metal selected from the group consisting of copper, zinc, and manganese. The blend can be a particulate blend or a blend carried by a liquid.

### DETAILED DESCRIPTION OF THE PREFERRED EMBODIMENT(S)

Before the present invention is disclosed and described, it is to be understood that this invention is not limited to the particular process steps and materials disclosed herein because such process steps and materials may vary somewhat. It is also to be understood that the terminology used herein is used for the purpose of describing particular embodiments only. The terms are not intended to be limiting because the scope of the present invention is intended to be limited only by the appended claims and equivalents thereof.

It is to be noted that, as used in this specification and the appended claims, the singular forms “a,” “an,” and “the” include plural referents unless the context clearly dictates otherwise.

The term “naturally occurring amino acid” or “traditional amino acid” shall mean amino acids that are known to be used for forming the basic constituents of proteins, including alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and combinations thereof.

The term “amino acid chelate” is intended to cover both the traditional definitions and the more modern definition of chelate as cited previously. Specifically, with respect to chelates that utilize traditional amino acid ligands, i.e., those used in forming proteins, chelate is meant to include metal ions bonded to proteinaceous ligands forming heterocyclic rings. Between the carboxyl oxygen and the metal, the bond can be covalent or ionic, but is preferably coordinate covalent. Additionally, at the a-amino group, the bond is typically a coordinate covalent bond. Proteinates of naturally occurring amino acids are included in this definition, as long as the proteinate includes naturally occurring amino acids. Complexes that do not form heterocyclic ring structures are specifically excluded from this definition, and as such, are outside the scope of the term “chelate.”

The term “proteinate” when referring to a metal proteinate is meant to include compounds where the metal is chelated to a hydrolyzed or partially hydrolyzed protein forming a heterocyclic ring. Coordinate covalent bonds, covalent bonds and/or ionic bonding may be present in the chelate structure, as long as a ring structure is present. As used herein, proteinates are included when referring to amino acid chelates.

The term “nutritionally relevant metal” is meant to mean any divalent (and in some embodiments, trivalent) metal that can be used as part of a nutritional supplement, is known to be beneficial to humans, and is substantially non-toxic when administered in traditional amounts, as is known in the art. Examples of such metals include copper, zinc, manganese, iron, chromium, cobalt, calcium, magnesium, and the like. In accordance with embodiments of the present invention, amino acid chelates that include copper, zinc, or manganese are especially preferred for use in alleviating or reducing alcohol dependency. However, in one embodiment, amino acid chelates or even non-chelate complexes that include other nutritionally relevant metals can be coadministered with copper, zinc, and/or manganese amino acid chelates.

With these definitions in mind, various methods and compositions are disclosed herein that are beneficial in reducing alcohol dependency and/or desire in humans. Specifically, a method for reducing alcohol desire or physical dependency in a human can comprise the steps of administering an amino acid chelate to a human having alcohol dependency symptoms or an unwanted desire for alcohol, wherein the amino acid chelate comprises a naturally occurring amino acid ligand and a metal selected from the group consisting of copper, zinc, and manganese, and wherein the amino acid to metal molar ratio is from 1:1 to 4:1. Metal proteinates having any one of the same metals can also be used, and are included within the definition of amino acid chelates.

In another embodiment, a composition for reducing alcohol dependency in humans can comprise a blend of a first amino acid chelate and a second amino acid chelate, wherein the first amino acid chelate and the second amino acid chelate each comprise a different metal selected from the group consisting of copper, zinc, and manganese. In a further detailed aspect of an embodiment of the invention, three metal amino acid chelates can be present in the particulate blend, wherein one comprises zinc, a second comprises copper, and a third comprises manganese. The blend can be a particulate blend which optionally includes a solid carrier, or the blend can be carried by a liquid.

With respect to both the method and composition embodiments, the naturally occurring amino acid ligand can be selected from the group consisting of alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and combinations thereof, including dipeptides and tripeptides formed by any combination of said amino acids. In a more detailed aspect, though any of the above traditional amino acid ligands can be used effectively, amino acid ligands having anti-oxidant properties or other alcohol dependency reduction properties can be preferred for use in some embodiments. Such amino acids include those selected from the group consisting of cystine, cysteine, arginine, histidine, lysine, glycine, and glutamic acid, and combinations thereof.

Regarding the method embodiment described, one of copper, zinc, or manganese can be used as the sole metal selected for use having one or more amino acid ligands chelated to the single metal. Alternatively, a combination of at least two metals can be present in an amino acid chelate cocktail formulation. Thus, any two of copper, zinc, and manganese can be selected for use. Again, with this embodiment, one or more ligand(s) can be independently chelated to each of the metals. In yet another embodiment, all three metals can be present in a composition mixture including a copper amino acid chelate, a zinc amino acid chelate, and a manganese amino acid chelate. Again, one or more ligands can be used to chelate the metals.

In embodiments where multiple amino acid chelates are present as part of a liquid (suspension or solution) or particulate dosage, or which are mixed with a liquid or solid carrier, the amino acid chelates present can be divided into various ratios. For example, if a zinc amino acid chelate is mixed with a copper amino acid chelate, of the total amino acid chelate present, the copper amino acid chelate to zinc amino acid chelate can be present at a weight ratio from about 40:1 to 1:40, with a preferred range being from about 5:1 to 1:5. The same ratios would also be present in embodiments where other amino acid chelates are prepared in a mixed batch, i.e., zinc/manganese or copper/manganese. When all three metals are present as amino acid chelates, then each metal amino acid chelate compared to the remaining two metal amino acid chelates should be present at a weight ratio of at least 1:20, e.g., zinc amino acid chelate to copper and manganese amino acid chelates should be at least 1:20. These ratios are given with respect to the presence of certain amino acid chelates without regard to weight variation of various amino acid ligands. With each embodiment, as stated previously, one or more amino acid ligands can be used for the amino acid chelates described. However, since the ratios given are by weight, one can consider the molecular weight of each amino acid ligand, as well as the molecular weight of the metals, when determining appropriate weight ratios.

Some reasons that copper, zinc, and manganese are selected as metals for use to reduce alcohol desire and/or alcohol dependency in humans and other mammals is due to the fact that ethanol abuse is believed to exacerbate deficiencies of these metals, particularly with respect to copper and zinc. For example, copper is believed to be involved in several biochemical abnormalities observed in alcoholic liver injury. Further, copper deficiency may affect scavenging of free radicals, thus leading to oxidative injury. Low zinc status has been observed in 30% to 50% of alcoholics. Alcohol decreases the absorption of zinc and increases loss of zinc in urine. In addition, many alcoholics do not eat an acceptable variety or amount of food, so their dietary intake of zinc may be inadequate. Additionally, improper levels of manganese can also be linked to alcohol desire or dependency. With these deficiencies, as ethanol metabolism becomes altered, frequently, an increase in alcohol intake results, whether by merely increased desire or dependency. Additionally, with respect to embodiments where one or more of the amino acid ligands used has anti-oxidant properties, the presence of free radicals in the body associated with alcohol metabolism and subsequent alcohol dependency can be reduced. Because these metals and amino acids are administered in the form of amino acid chelates, the metals are delivered in a form that is more bioavailable than the same metals when administered as metal salts. Thus, these metals in the form of amino acid chelates can have a greater therapeutic effect than when similar amounts of the same metals are administered in salt form.

This being said, the purpose of the invention is not to describe any specific mechanism as to why these compositions reduce alcohol desire or dependency in humans, only that the compositions and methods that are in accordance with an embodiment of the present invention reduce such alcohol desire or dependency.

In one embodiment, the amount of the amino acid chelate(s) delivered at a single administration or dosage can be at or above the recommended daily allowance (RDA) for the mineral being delivered. In another embodiment, an amount less than the RDA can be delivered, particularly when more than one of copper, zinc, and/or manganese is being delivered to the subject. The RDA for copper is generally from 1.5 mg to 3 mg (US) and 1.2 mg (EU). Thus, in one embodiment where more than the RDA is delivered, copper can be delivered at from 1.5 mg/day to 50 mg/day. The RDA for zinc is 11 mg for males and 8 mg for females (US) and 15 mg (EU). Thus, in one embodiment where more than the RDA is delivered, zinc can be delivered at from 8 mg/day to 100 mg/day. The RDA for manganese is 2.5 mg to 7 mg (US) with no official EU figures. Thus, in one embodiment where more than the RDA is delivered, manganese can be delivered at from 2.5 mg/day to 50 mg/day. As can be seen, in order to alleviate alcohol dependency, increasing the delivered amounts to greater than the RDA can be beneficial in some circumstances. However, these ranges are exemplary only, and extended periods of delivery of large amounts of any given metal should be considered on a case by case basis. On the other hand, as amino acid chelates provide a more bioavailable source of these metals, lower amounts of the metals can be administered with greater body usage and less metal waste passing through the gastrointestinal tract unused. Thus, copper can be delivered at less than 1.5 mg/day, zinc can be delivered at less than 8 mg/day, and manganese can be delivered at less than 2.5 mg/day with positive results in some circumstances.

It is not the purpose of the present invention to describe how to prepare amino acid chelates that can be used with the present invention. Any amino acid chelate or combination of chelates comprising copper, zinc, and/or manganese can be used with varying degrees of effectiveness. Suitable methods for preparing such amino acid chelates can include those described in U.S. Pat. No. 4,830,716, for example. However, combinations of such chelates as part of a particulate composition for reducing alcoholic desire and/or dependency are included as an embodiment of the present invention.

### EXAMPLES

The following examples are illustrative of a present method of reducing alcohol dependency in humans, as well as compositions that can be used for the same. As such, the following examples should not be considered as limitations of the present invention, but merely demonstrate the effectiveness of the methods and compositions described herein.

### Example 1

### Reduction of Alcohol Dependency in Laboratory Rats

Preparative Procedures and Conditions

Ten male Sprague Dawley albino rats of similar age and weight were individually caged in an environment maintained at 20° C. The rats were randomly separated into a control group and a treatment group (5 rats in each group). All rats were fed dry laboratory rat chow ad libitum, and the basal diet of each rat contained 20 ppm zinc carbonate, 15 ppm copper carbonate, and other essential nutrients. Additionally, each rat cage was fitted with two bottles, one containing water, and the other containing a mixture of 5% ethanol and 95% water (v/v). All of the water used was distilled and deionized. The positions of the water bottles on each cage were changed daily to reduce the likelihood of each rat learning the locations of the different bottles, and thus, influence the selection of which bottle the rat chose to drink from. All rats had access to both the water and the ethanol/water bottle throughout the entire study ad libitum.

Phase 1—Developing Alcohol Dependency in Rats

All of the rats were intragastrically administered a 1:1 volume of water and ethanol in an amount such that 8 g of ethanol per kg of body weight was received. This amount is equivalent to about ⅔ of the lethal dose for rats. Such a dosage was administered every morning at the same time for a 28 day period, which created a physical need for ethanol in each of the rats. See, Liubimov BI et al., Chronic alcoholic intoxications in animals or a model for studying the safety of new anti-alcoholic agents (abstract), Farmakol Toksiol 46:98-102, Physiological Abstract 9010 (1983). Each rat was weighed once a week on an electronic balance and the daily quantity of administered ethanol was adjusted according to each rat's individual weekly weight. Because all of the rats were approximately the same size, each received approximately 2.2 ml of ethanol per day.

Phase 2—Study of Effects of Amino Acid Chelates on Alcohol Dependency in Rats

After 28 days (Phase 1), the intragastric administration of ethanol was discontinued with all of the rats, both from the control and treatment group. While familiar to the ethanol/water dispensing bottle and the water-dispensing bottle described previously, each rat had to seek out the ethanol/water-dispensing bottle as its sole source of ethanol.

At day 29, each of the 5 rats in the treated group received a daily dose of zinc, manganese, and copper amino acid chelates (metal bisglycinates) dissolved in distilled and deionized water. About 100 μg (approximately 0.49 mg/kg body wt) of zinc, 100 μg of manganese (approximately 0.49 mg/kg body wt) and 2 μg copper (approximately 0.01 mg/kg body wt) was present in the water. The diluted mineral solution was administered daily to each rat in the treated group intragastrically using a similar method described in phase 1. This phase continued for a total of 21 days.

Regarding the control group, even though they received intrinsic copper, manganese, and zinc salts as part of the rat chow formulation, they did not receive any copper, manganese, or zinc amino acid chelates during any phase of the study.

With respect to both the control group and the treatment group, beginning on day 29, the quantities of both the ethanol/water mixture (Et-OH/H₂0) and the water (H₂O) consumed in each 24 hour period were measured and recorded, as is shown in Table 2 below:

TABLE 2

| | CONTROL GROUP | | TREATED GROUP | |
|---|---|---|---|---|
| Day | H₂O | Et—OH/H₂O | H₂O | Et—OH/H₂O |
| (phase 2) | (ml/rat) | (ml/rat) | (ml/rat) | (ml/rat) |
| 29 | 0 | 0 | 0 | 0 |
| 30 | 43.0 | 8.3 | 50 | 0 |
| 31 | 21.3 | 14.0 | 35.3 | 1.5 |
| 32 | 8.3 | 7.0 | 20.0 | 0.75 |
| 33 | 9.3 | 7.6 | 25.0 | 1.0 |
| 34 | 7.2 | 7.1 | 18.0 | 0.75 |
| 35 | 3.2 | 9.0 | 36.5 | 1.25 |
| 36 | 10.2 | 7.2 | 33.0 | 1.0 |
| 37 | 15.0 | 10.3 | 25.0 | 1.0 |
| 38 | 10.2 | 13.0 | 35.5 | 1.0 |
| 39 | 15.5 | 22.0 | 45.5 | 1.0 |
| 40 | 14.5 | 14.0 | 29.0 | 1.0 |
| 41 | 22.5 | 11.0 | 41.0 | 0.5 |
| 42 | 28.0 | 11.0 | 40.5 | 2.0 |
| 43 | 20.0 | 12.5 | 35.0 | 0.5 |
| 44 | 20.5 | 9.5 | 38.0 | 1.0 |
| 45 | 15.0 | 13.0 | 37.5 | 0.5 |
| 46 | 29.5 | 9.5 | 30.0 | 1.0 |
| 47 | 19.0 | 15.0 | 36.0 | 0.5 |
| 48 | 30.0 | 11.5 | 36.0 | 2.0 |
| 49 | 14.5 | 14.5 | 32.5 | 0.5 |

Table 3 below shows the mean weights and liquid consumption of the rats from both the control group and the treated group.

TABLE 3

| | CONTROL GROUP | TREATED GROUP |
|---|---|---|
| | Mean Wt/Liq Con | Mean Wt/Liq Con |
| Initial Weight | 191.1 g/21.8 g | 189.6 g/24.0 g |
| Terminal Weight | 233.8 g/24.3 g | 210.9 g/13.8 g |
| H₂O consumption/day | 12.9 ml/9.5 ml | 33.9 ml/7.9 ml |
| (Phase 2) | | |
| ethanol consumption/day | 2.3 ml/0.72 ml | 0.1 ml/0.02 ml |
| (Phase 2) | | |
| ethanol/H₂O consumption/day | 11.4 ml/3.6 ml | 0.9 ml/0.5 ml |
| (Phase 2) | | |
| total liquid consumption/day | 24.3 ml/10.3 ml | 34.8 ml/7.8 ml |
| (Phase 2) | | |

Results

By the end of Phase 1 (the first 28 day period), all of the rats in both groups exhibited behavior suggesting ethanol abuse. All animals preferred drinking from the ethanol/water bottle over the water bottle during Phase 1.

As can be seen from Tables 2 and 3, the rats that were supplemented with zinc and copper amino acid chelates (the treated group) exhibited a significantly reduced ethanol/water consumption in phase 2 as compared to the control group. Further, in phase 2, the water consumption among the treated group was much greater than among the control group. This shows that, in rats with confirmed ethanol abuse, there was significantly less desire to consume ethanol when the animals received zinc and copper as amino acid chelates.

### Example 2

### Preparation of Admixture of Copper Bisarginate and Zinc Bisarginate

An amino acid chelate containing a particulate mixture was prepared comprising about 45 μg (0.22 mg/kg body wt) of a copper bisarginate and 100 μg (0.49 mg/kg body wt) of a zinc bisarginate. This formulation, when administered in an oral dosage form, or with a carrier, provides reduced alcohol dependency and/or desire among alcohol dependent animals.

### Example 3

### Preparation of Admixture of Copper Bisglycinate, Zinc Bisglycinate, and Manganese Bisglycinate

An amino acid chelate containing a particulate mixture was prepared comprising about 20 mg of a copper bisglycinate, 30 mg of a zinc bisglycinate, and 20 mg of a manganese bisglycinate. This formulation, when administered as an oral dosage tablet or in another similar form, or with a carrier, provides reduced alcohol dependency and/or desire among alcohol dependent humans.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A method for reducing alcohol desire or dependency, comprising the steps of:
administering a therapeutically effective amount of an amino acid chelate to a human having symptoms of alcohol desire or dependency, said amino acid chelate comprising a naturally occurring amino acid ligand and a metal selected from the group consisting of copper, zinc, and manganese, and wherein the amino acid to metal molar ratio is from 1:1 to 4:1.

2. A method as in claim 1, wherein the metal is copper.

3. A method as in claim 1, wherein the metal is zinc.

4. A method as in claim 1, wherein the metal is manganese.

5. A method as in claim 1, further comprising coadministering to the human a second amino acid chelate that is different than the amino acid chelate, said second amino acid chelate also comprising a naturally occurring amino acid ligand and a metal selected from the group consisting of copper, zinc, and manganese, and wherein the second amino acid chelate has an amino acid to metal molar ratio from 1:1 to 4:1.

6. A method as in claim 1, wherein the amino acid is selected from the group consisting of glycine, cystine, cysteine, arginine, histidine, lysine, glutamic acid, and combinations thereof.

7. A method as in claim 5, further comprising coadministering to the human a third amino acid chelate that is different than the amino acid chelate and the second amino acid chelate, said third amino acid chelate also comprising a naturally occurring amino acid ligand and a metal selected from the group consisting of copper, zinc, and manganese, and wherein the third amino acid chelate has an amino acid to metal molar ratio from 1:1 to 4:1.

8. A method as in claim 1, wherein the step of administering includes coadministering a zinc amino acid chelate with a copper amino acid chelate.

9. A method as in claim 1, wherein the step of administering includes coadministering a zinc amino acid chelate with a manganese amino acid chelate.

10. A method as in claim 1, wherein the step of administering includes coadministering a manganese amino acid chelate with a copper amino acid chelate.

11. A method as in claim 1, wherein the step of administering includes coadministering a zinc amino acid chelate with a copper amino acid chelate and a manganese amino acid chelate.

12. A method as in claim 1, wherein the step of administering includes coadministering a first amino acid chelate having a first amino acid with a second amino acid chelate having a second amino acid that is different than the first amino acid.

13. A method as in claim 12, wherein the first amino acid chelate is a zinc amino acid chelate and the second amino acid chelate is a copper or manganese amino acid chelate.

14. A method as in claim 12, wherein the first amino acid chelate and the second amino acid chelate includes the same metal.

15. A method as in claim 1, wherein the therapeutically effective amount of the amino acid chelate includes at least one amino acid chelate that is administered within a composition, wherein the at least one amino acid chelate provides the most significant benefit with respect to reducing alcohol desire or dependency when delivered alone compared to any other single component within the composition when also delivered alone.

16. A composition for reducing alcohol dependency in humans, comprising a blend of:
a) a first amino acid chelate comprising a naturally occurring amino acid and a first metal selected from the group consisting of copper, zinc, and manganese; and
b) a second amino acid chelate comprising a second naturally occurring amino acid and a second metal selected from the group consisting of copper, zinc, and manganese, wherein the first amino acid chelate and the second amino acid chelate each having an amino acid to metal molar ratio from 1:1 to 4:1, and wherein the first amino acid chelate and the second amino acid chelate are different.

17. A composition as in claim 16, wherein the first metal and the second metal are different.

18. A composition as in claim 16, wherein the first naturally occurring amino acid and the second naturally occurring amino acid are different.

19. A composition as in claim 16, wherein the first amino acid chelate is a copper amino acid chelate, the second amino acid chelate is a zinc amino acid chelate, and wherein the blend further comprises a third amino acid chelate comprising a naturally occurring amino acid and manganese, said third amino acid chelate having an amino acid to metal molar ratio from 1:1 to 4:1.

20. A composition as in claim 16, wherein the blend is a particulate blend.

21. A composition as in claim 20, wherein the particulate blend is formulated within a tablet or capsule.

22. A composition as in claim 16, wherein the blend is within a liquid carrier.
